(19) United States
(12) Patent Application Publication
(10) Pub. No.: US 2023/0321339 A1
(43) Pub. Date: Oct. 12, 2023

(54) INFUSION PUMP

(71) Applicant: TERUMO KABUSHIKI KAISHA, Tokyo (JP)

(72) Inventors: Shun DEMIZU, Hadano-shi (JP); Shun HACHIMURA, Hadano-shi (JP); Mio SHOUJI, Naka-gun (JP)

(73) Assignee: TERUMO KABUSHIKI KAISHA, Tokyo (JP)

(21) Appl. No.: 18/209,120

(22) Filed: Jun. 13, 2023

Related U.S. Application Data
(63) Continuation of application No. PCT/JP2021/037214, filed on Oct. 7, 2021.

(30) Foreign Application Priority Data
Dec. 16, 2020 (JP) 2020-208585

(51) Int. Cl. A61M 5/142

(52) U.S. Cl. CPC A61M 5/142

(57) ABSTRACT

An infusion pump for feeding a drug, the infusion pump includes: a main body portion comprising a first engagement portion; and a main body mounting portion comprising a second engagement portion configured to engage with the first engagement portion. The main body portion and the main body mounting portion are configured to feed a drug in an infusion tube by sandwiching the infusion tube therebetween. The main body mounting portion is mounted on the main body portion by engaging the first engagement portion and the second engagement portion. The first engagement portion and the second engagement portion are engaged with each other by deformation or movement of the first engagement portion or the second engagement portion by elastic force in conjunction with a mounting operation in which the main body portion and the main body mounting portion approach each other with the infusion tube sandwiched therebetween.

### CROSS-REFERENCE TO RELATED APPLICATIONS

This is a bypass continuation of PCT/JP2021/037214, filed on Oct. 7, 2021, which claims priority to Japanese Patent Application No. 2020-208585, filed on Dec. 16, 2020. The contents of these applications are hereby incorporated by reference in their entirety.

### BACKGROUND

The present disclosure relates to an infusion pump.

A patient controlled analgesia (PCA) pump includes a drive unit that feeds liquid and a storage unit that stores a drug.

In a case where a PCA pump is used to administer medical anesthesia to a patient, it must be provided with a structure that prevents the patient or the patient's family from taking out the drug according to regulations (see PCT Publication No. WO 2011/068648 A2 (“Patent Literature 1”).

### SUMMARY

In order to realize such a structure, the pump described in Patent Literature 1 is provided with a lock mechanism that fixes the drug storage unit when the drug storage unit is attached to the drive unit and releases the drug storage unit when the drug storage unit is removed from the drive unit. However, the lock mechanism is complicated because it requires many operations. In addition to the PCA pump, there is room for improvement in the operability of the lock mechanism even for other pumps.

In view of the above, an object of the present disclosure is to provide an infusion pump including a lock mechanism capable of realizing a locked state by fewer operations than the lock mechanism according to the prior art.

According to one embodiment of the present disclosure, an infusion pump capable of feeding a drug includes a main body portion and a main body mounting portion capable of feeding a drug in an infusion tube by sandwiching the infusion tube between. The main body portion includes a first engagement portion, the main body mounting portion includes a second engagement portion engaged with the first engagement portion, the main body mounting portion is mounted on the main body portion by engaging the first engagement portion and the second engaged with each other, and the first engagement portion and the second engagement portion are engaged with each other by deformation or movement of the first engagement portion or the second engagement portion by elastic force in conjunction with a mounting operation in which the main body portion and the main body mounting portion approach each other with the infusion tube sandwiched between.

According to one aspect of the present invention, the first engagement portion and the second engagement portion is deformed or moved by an elastic force in a direction intersecting a moving direction of the main body portion and the main body mounting portion in the mounting operation.

According to one aspect of the present invention, one engagement portion of the first engagement portion and the second engagement portion includes a claw portion engaged with the other engagement portion, an endless portion to which the claw portion is attached, a shaft penetrating the endless portion, and an elastic portion capable of biasing the endless portion in a longitudinal direction of the shaft. The one engagement portion is engaged with the other engagement portion by the claw portion and the endless portion moving in the longitudinal direction of the shaft by an elastic force of the elastic portion.

According to one aspect of the present invention, an elongated hole extending in a direction inclined with respect to the longitudinal direction is formed in the endless portion. The shaft includes a protrusion portion inserted into the elongated hole, and the endless portion moves in the longitudinal direction by rotating relative to the shaft.

According one aspect of the present invention, the first engagement portion and the second engagement portion can be changed in form from an engagement state to an engagement release state by the endless portion rotating relative to the shaft and moving in the longitudinal direction. The main body portion includes a third engagement portion that engages the second engagement portion when the first engagement portion and the second engagement portion are changed from the engagement state to the engagement release state. The third engagement portion allows the main body mounting portion to be separated from the main body portion by moving or being deformed in the longitudinal direction.

According to one aspect of the present invention, the first engagement portion includes a housing provided with an opening portion extending in a direction perpendicular to a moving direction of the main body mounting portion in the mounting operation. The second engagement portion includes an insertion portion provided with a concave portion in a direction perpendicular to the moving direction, and a movement portion capable of varying a protrusion amount from the concave portion by moving in the concave portion. The second engagement portion is engaged with the first engagement portion by the movement portion varying a protrusion amount from the concave portion in the mounting operation and entering the opening portion of the housing.

According to one aspect of the present invention, the first engagement portion further includes a movement engagement portion that is movable in a moving direction of the movement portion and is engageable with the movement portion, and a pushing portion that can push out the movement engagement portion toward the movement portion. The first engagement portion and the second engagement portion are released by the pushing portion pushing out the movement engagement portion toward the movement portion and the movement portion moving out of the opening portion of the housing.

According to one aspect of the present invention, the main body portion includes a third engagement portion that engages the second engagement portion when the first engagement portion and the second engagement portion are changed from the engagement state to the engagement release state. The third engagement portion allows the main body mounting portion to be separated from the main body portion by moving or being deformed in the moving direction of the movement portion.

According to certain embodiments of the infusion pump of the present disclosure, the first engagement portion and the second engagement portion are engaged with each other by being deformed or moved by an elastic force when the main body portion and the main body mounting portion are mounted, in a manner that it is possible to realize a lock mechanism capable of realizing a locked state by fewer operations than the lock mechanism according to the prior art.

### BRIEF DESCRIPTION OF THE DRAWINGS

FIG. 1 is a front view of an infusion pump according to a first embodiment.

FIG. 2 is a bottom view of the infusion pump of FIG. 1.

FIG. 3 is a view illustrating an unlocked state in which a lock mechanism of the infusion pump of FIG. 1 is released.

FIG. 4 is a perspective view of a main body portion of FIG. 1.

FIG. 5 is a perspective view of a main body mounting portion of FIG. 1.

FIG. 6 is a view of an internal structure of the infusion pump of FIG. 1.

FIG. 7 is a view of an internal structure of the infusion pump of FIG. 1.

FIG. 8 is a view of an internal structure of the infusion pump of FIG. 1.

FIG. 9 is a view of an internal structure of the infusion pump of FIG. 1.

FIG. 10 is an enlarged view of an engagement portion of the main body portion of FIG. 1.

FIG. 11 is an enlarged perspective view of the engagement portion of the main body portion of FIG. 1.

FIG. 12 is an enlarged perspective view of the engagement portion of the main body portion of FIG. 1.

FIG. 13 is a view illustrating a liquid feeding mechanism of the main body portion of FIG. 1.

FIG. 14 is a view illustrating a buffer mechanism of an infusion tube according to the first embodiment.

FIG. 15 is a view illustrating the buffer mechanism of the infusion tube according to the first embodiment.

FIGS. 16(a)-16(d) are views illustrating an engagement mechanism according to a second embodiment.

### DETAILED DESCRIPTION

### First Embodiment

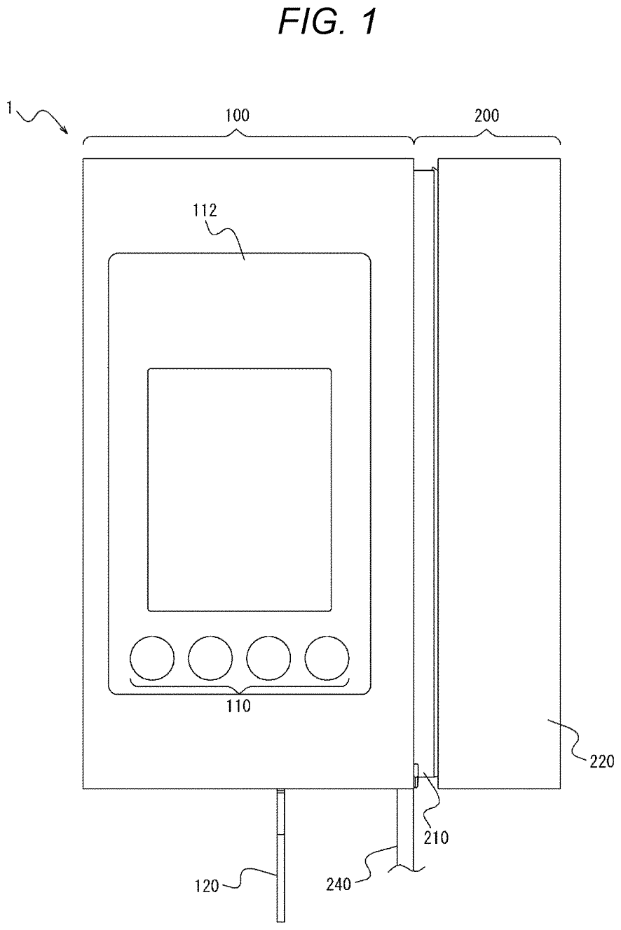

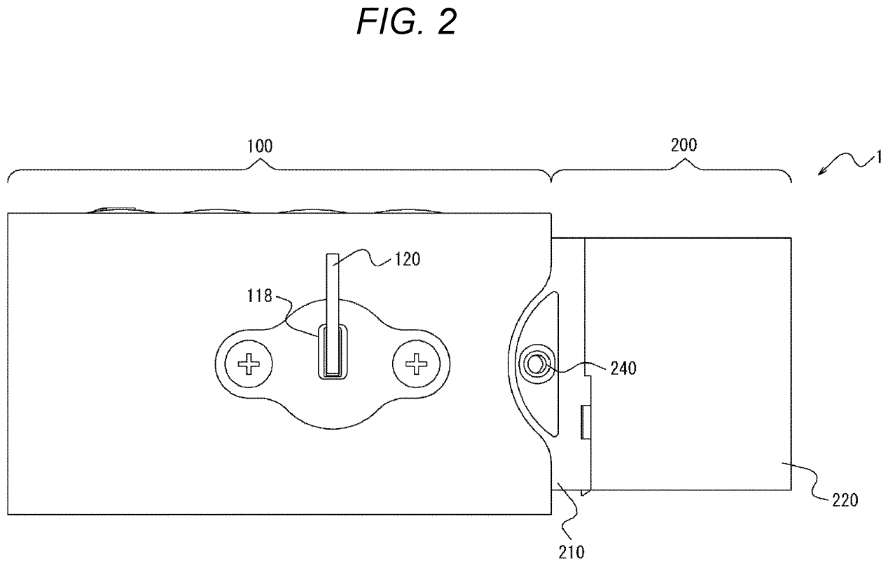

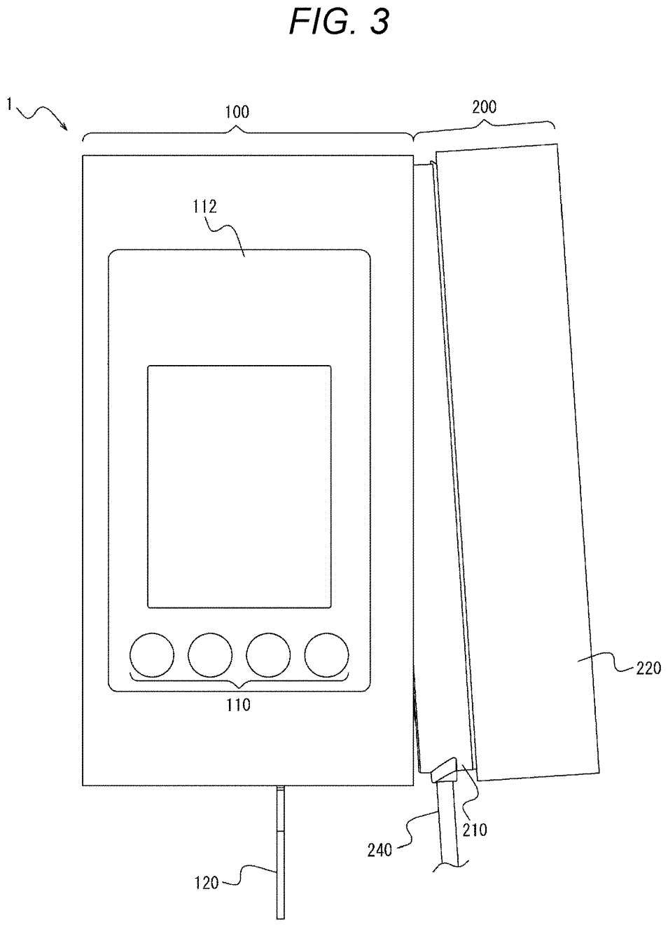

FIGS. 1 to 3 illustrate an infusion pump 1 according to a first embodiment. FIGS. 1 and 2 illustrate a locked state in which the lock mechanism of the infusion pump 1 is working, and FIG. 3 illustrates an unlocked state in which the lock mechanism of the infusion pump 1 is released.

The infusion pump 1 includes a main body portion 100 and a drug filling unit 200. The main body portion 100 includes an operation unit 110 and a display unit 112. The operation unit 110 is used to operate the infusion pump 1. In the present embodiment, the operation unit 110 is a button, but any other operation means may be used. The display unit 112 displays the state of the infusion pump 1. In the present embodiment, the display unit 112 is a liquid crystal display (LCD), but any other display means may be used, and the display unit may have a function capable of performing an input operation such as a touch panel. In the infusion pump 1, a state in which the drug filling unit 200 is mounted to the main body portion 100 (hereinafter, it is simply referred to as a “locked state”) and a state in which the drug filling unit 200 can be separated from the main body portion 100 (hereinafter, it is simply referred to as an “unlocked state”) can be realized by the lock mechanism. The main body portion 100 is provided with a key hole 118 into which a key 120 is inserted. The lock mechanism between the main body portion 100 and the drug filling unit 200 is released by rotating the key 120 inserted into the key hole 118 in a predetermined direction.

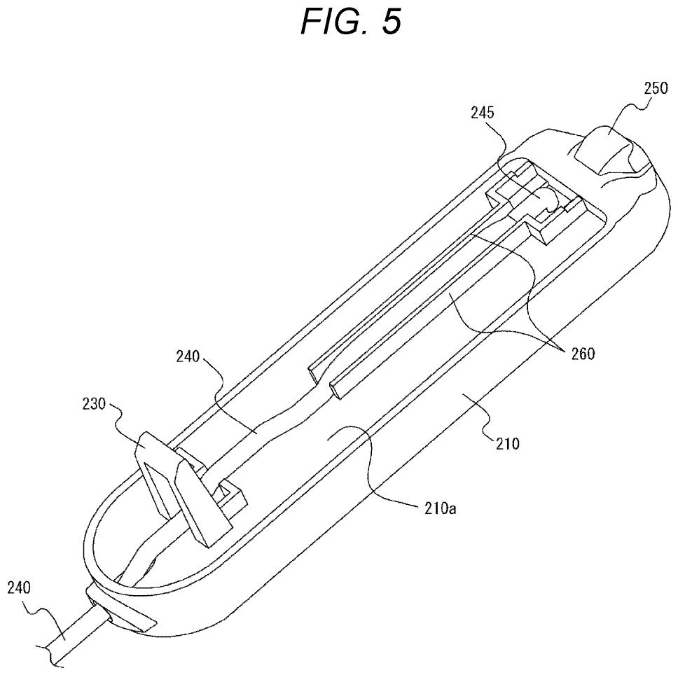

The drug filling unit 200 includes a main body mounting portion 210 attached to the main body portion 100, a drug storage unit 220 attached to the main body mounting portion 210, and an infusion tube 240 fixed to the main body mounting portion 210 and communicating with the drug storage unit 220. The main body mounting portion 210 is attachable to and detachable from the main body portion 100. The main body mounting portion 210 includes a tube receiving surface 210a (see FIG. 5) facing the main body portion 100 when mounted to the main body portion 100. The infusion tube 240 is fixed on the tube receiving surface 210a of the main body mounting portion 210. The drug storage unit 220 is used to store a drug. The infusion tube 240 is used as a flow path when feeding a drug stored in the drug storage unit 220 to supply the drug to a patient.

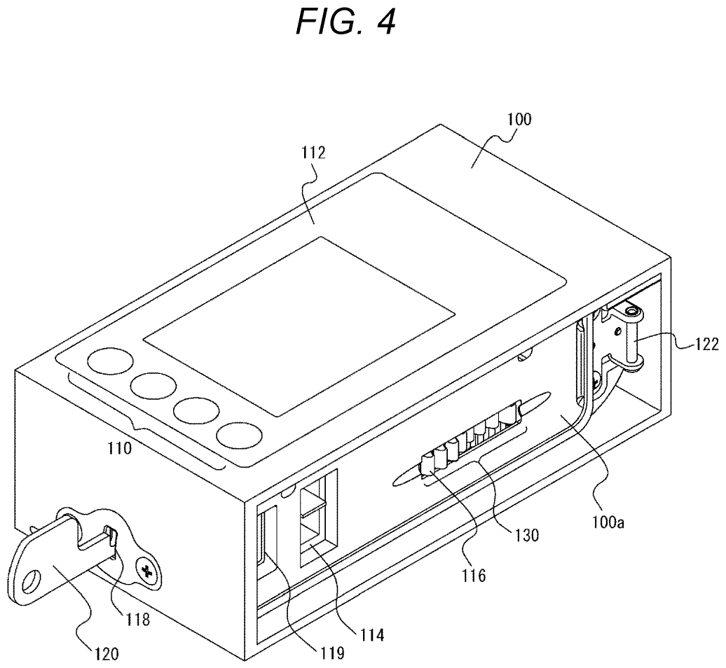

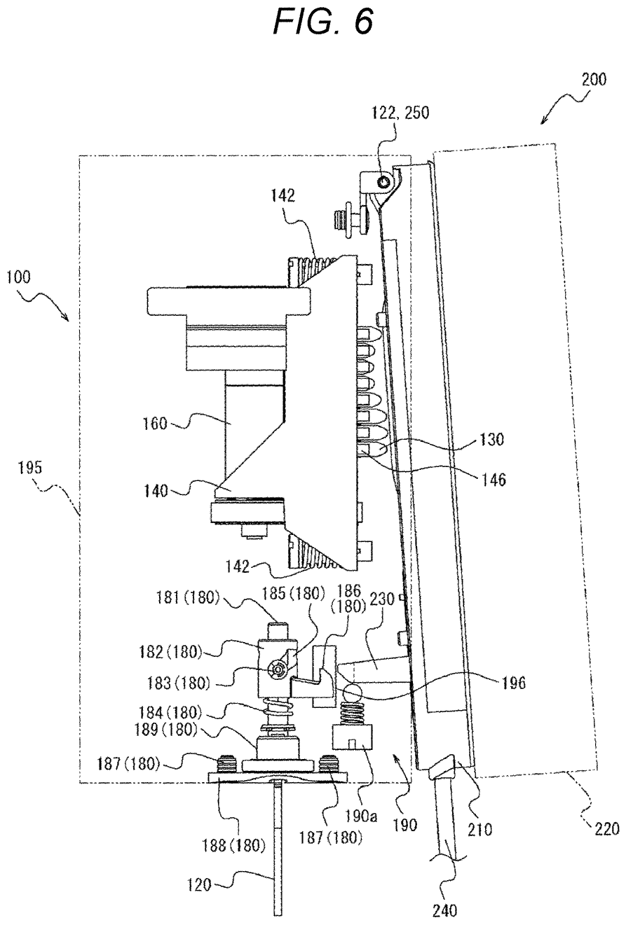

FIG. 4 is a perspective view of the main body portion 100. The main body portion 100 includes an insertion hole 119 for attaching the main body mounting portion 210, a finger portion 130, a finger portion hole 116 for exposing the finger portion 130, a sensor hole 114 for performing detection by a sensor, and a pivotal attachment portion 122 for enabling pivotal attachment to the main body mounting portion 210 on a main body facing surface 100a facing the tube receiving surface 210a (see FIG. 5) of the main body mounting portion 210. The main body portion 100 and the main body mounting portion 210 sandwich the infusion tube 240 between the main body facing surface 100a and the tube receiving surface 210a in a locked state. The main body portion 100 and the main body mounting portion 210 can be mounted by performing an operation of approaching each other with the infusion tube sandwiched between (hereinafter, it is simply referred to as “mounting operation”) and engaging a first engagement portion 180 (see FIG. 6 and the like) to be described later of the main body portion 100 and a second engagement portion 230 (see FIG. 6 and the like) to be described later of the main body mounting portion 210.

The finger portion 130 controls the flow rate of the drug by pressing the infusion tube 240 sandwiched between the finger portion 130 and the main body mounting portion 210. The insertion hole 119 enables insertion of the second engagement portion 230 (see FIG. 5) to be described later of the main body mounting portion 210 when the main body mounting portion 210 is mounted to the main body portion 100. The sensor hole 114 enables monitoring of the flow of the drug by a sensor such as a blockage sensor, for example, but may be covered with a transparent plastic material and the like depending on the type of sensor, and is appropriately installed. The pivotal attachment portion 122 enables pivotal attachment of the main body mounting portion 210 to a grip portion 250 (see FIG. 5) to be described later.

FIG. 5 is a perspective view of the main body mounting portion 210. The main body mounting portion 210 includes the second engagement portion 230 to be described in detail later, a tube connector 245 that connects and communicates the infusion tube 240 and the drug storage unit 220, a tube guide portion 260 provided to sandwich the infusion tube 240, and the grip portion 250 provided at an end portion of the main body mounting portion 210 to be pivotally attached to the pivotal attachment portion 122 of the main body portion 100 on the tube receiving surface 210a facing the main body facing surface 100a. In the present embodiment, the second engagement portion 230 is a protrusion having an opening penetrating in the extending direction of the infusion tube 240, but may have another shape that can be engaged with the first engagement portion 180 (see FIGS. 6 to 9) to be described later. Therefore, for example, the opening of the second engagement portion 230 may be a concave portion that does not penetrate in the extending direction of the infusion tube 240.

The second engagement portion 230 is inserted into the insertion hole 119 of the main body portion 100 when the main body mounting portion 210 is mounted on the main body portion 100. The tube connector 245 and the infusion tube 240 serve as a flow path for supplying a drug stored in the drug storage unit 220 to a patient. The tube guide portion 260 restricts meandering of the infusion tube 240 by sandwiching the infusion tube 240 on the tube receiving surface 210a. The grip portion 250 is pivotally attached to the pivotal attachment portion 122 of the main body portion 100. As a result of the pivotal attachment, the drug filling unit 200 is pivotable with respect to the main body portion 100.

FIGS. 6 to 9 illustrate an internal structure of the infusion pump 1. The main body portion 100 includes, in addition to the insertion hole 119, the finger portion 130, the finger portion hole 116, the sensor hole 114, and the pivotal attachment portion 122 described above illustrated in FIG. 4, a drive unit 160 that drives the finger portion 130, a drive unit cover 140 that covers the drive unit 160, a finger guide 146 that is integrally formed with the drive unit cover 140 and controls a movement direction of the finger portion 130, an elastic portion 142 that biases the drive unit cover 140 in a direction toward the drug filling unit 200, the first engagement portion 180 that engages with the second engagement portion 230, and a ball plunger 190a as a third engagement portion 190 that restricts separation of the main body mounting portion 210 from the main body portion 100 in a case where a state changes from a locked state to an unlocked state.

The first engagement portion 180 includes a claw portion 186 that engages with the second engagement portion 230 when the main body mounting portion 210 is mounted to the main body portion 100, an endless portion 182 to which the claw portion 186 is attached, a shaft 181 that penetrates the endless portion 182, an elastic portion 184 capable of biasing the endless portion 182 in the longitudinal direction of the shaft 181, a shaft holding portion 189 that is provided under the elastic portion 184 and surrounds the shaft 181, and a housing attachment portion 188 that fixes the shaft holding portion 189 to a housing 195 of the main body portion 100. In the present embodiment, the endless portion 182 has a cylindrical shape, but may have another endless shape. The housing attachment portion 188 is provided with the key hole 118. The housing attachment portion 188 is attached to the housing 195 by a screw 187.

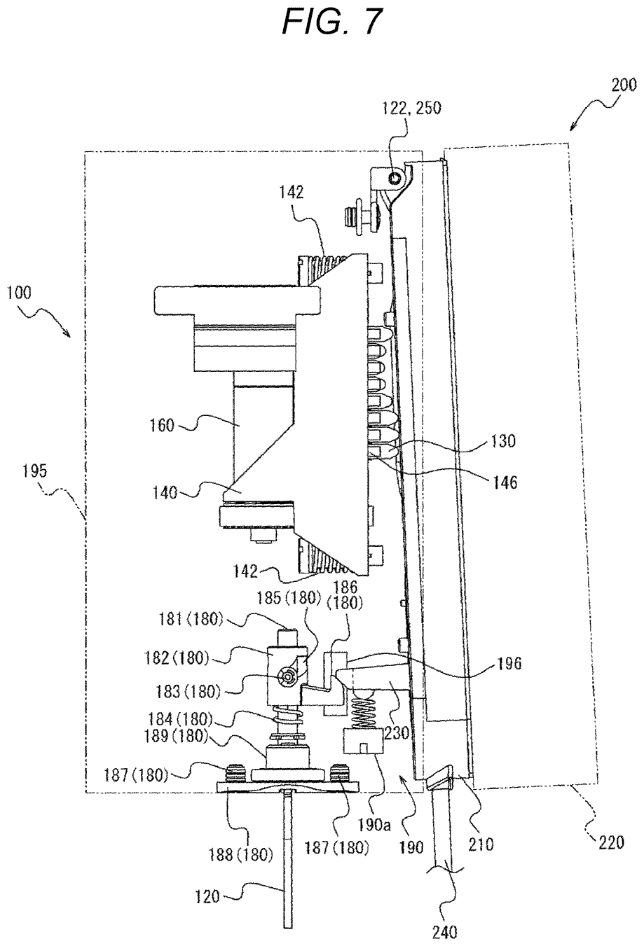

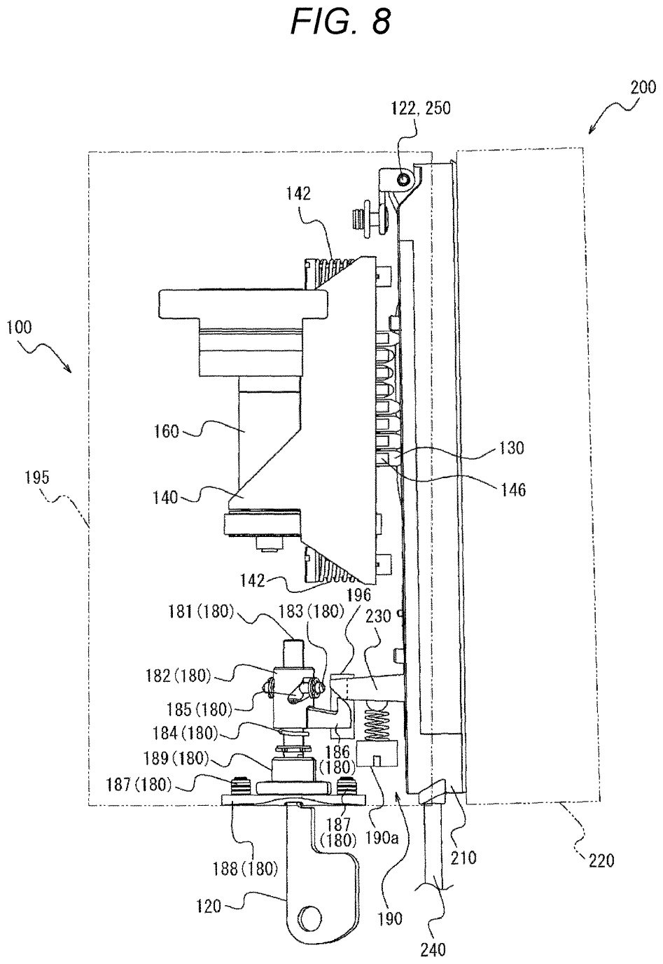

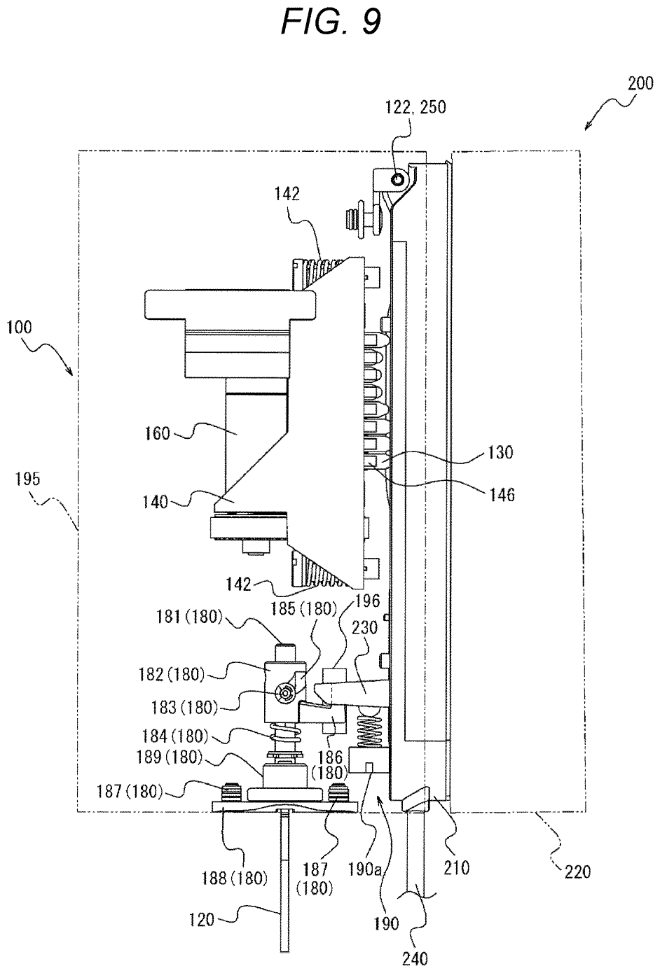

With reference to FIGS. 6 to 9, a procedure for mounting the drug filling unit 200 to the main body portion 100 will be described. In the state illustrated in FIGS. 6 to 8, the drug filling unit 200 is not completely mounted to the main body portion 100. In FIG. 6, the grip portion 250 is pivotally attached to the pivotal attachment portion 122, but the second engagement portion 230 is not in contact with the claw portion 186, and is only in contact with the ball plunger 190a as the third engagement portion 190. Note that the ball plunger 190a illustrated in FIG. 6 is pushed down and compressed by the second engagement portion 230, and is not fitted into the opening of the second engagement portion 230. FIG. 7 illustrates a state in which the mounting operation of moving the main body mounting portion 210 closer to the main body portion 100 from the state illustrated in FIG. 6 is advanced (the main body mounting portion 210 in FIG. 6 is moved to the left). In FIG. 7, the second engagement portion 230 is in contact with the claw portion 186. In addition, in FIG. 7, the pushing down of the ball plunger 190a by the second engagement portion 230 is released, and the ball plunger 190a is fitted into the opening of the second engagement portion 230. FIG. 8 illustrates a state in which the mounting operation of moving the main body mounting portion 210 closer to the main body portion 100 from the state illustrated in FIG. 7 is further advanced. In FIG. 8, the second engagement portion 230 slides on the claw portion 186 to push down the claw portion 186 to one side (lower side in FIGS. 6 to 9) in the longitudinal direction of the shaft 181. The claw portion 186 can be pushed down to one side in the longitudinal direction of the shaft 181 by elastically deforming and compressing the elastic portion 184. FIG. 9 illustrates a state in which the mounting operation of moving the main body mounting portion 210 closer to the main body portion 100 from the state illustrated in FIG. 8 is further advanced. In FIG. 9, the second engagement portion 230 climbs over the claw portion 186, and the first engagement portion 180 and the second engagement portion 230 are engaged. That is, a state in which attachment of the main body portion 100 and the main body mounting portion 210 is completed is illustrated. A part of the claw portion 186 illustrated in FIG. 9 is fitted into the opening of the second engagement portion 230. The claw portion 186 illustrated in FIG. 9 moves toward the other side (the upper side in FIGS. 6 to 9) in the longitudinal direction of the shaft 181 than the state illustrated in FIG. 8 by the restoring force of the elastic portion 184, and is fitted into the opening of the second engagement portion 230. In this manner, the first engagement portion 180 and the second engagement portion 230 are engaged with each other by the first engagement portion 180 or the second engagement portion 230 moving by the elastic force in conjunction with the mounting operation in which the main body portion 100 and the main body mounting portion 210 approach each other with the infusion tube 240 sandwiched between. In the present embodiment, when the claw portion 186 is pushed down, the endless portion 182 to which the claw portion 186 is attached also moves downward. However, for example, the claw portion itself formed of an elastic body may be elastically deformed to be displaced in the longitudinal direction of the shaft 181. That is, the first engagement portion 180 or the second engagement portion 230 may be deformed by elastic force to be engaged with each other.

As described above, in FIG. 9, the drug filling unit 200 is mounted to the main body portion 100. More specifically, when the opening provided in the second engagement portion 230 comes to the upper side of the claw portion 186 that has been pushed down, the endless portion 182 that is biased by the elastic portion 184 and is integrated with the claw portion 186 moves upward. As a result, the claw at the distal end that is a part of the claw portion 186 enters the opening of the second engagement portion 230. As a result, the first engagement portion 180 and the second engagement portion 230 are engaged with each other. As a result, mounting of the drug filling unit 200 and the main body portion 100 is completed. Note that the position of the claw portion 186 illustrated in FIG. 9 in the longitudinal direction of the shaft 181 is the same as the position illustrated in FIGS. 6 and 7. As described above, the first engagement portion 180 of the present embodiment is deformed or moved by the elastic force in the direction (in FIGS. 6 to 9, the longitudinal direction of the shaft 181 as an example) intersecting the moving direction (in FIGS. 6 to 9, the left-right direction) of the main body portion 100 and the main body mounting portion 210 in the mounting operation. In addition, in the present embodiment, the first engagement portion 180 of the main body portion 100 is deformed or moved by the elastic force to vary the engagement state with the second engagement portion 230 of the main body mounting portion 210, but the present invention is not limited to this configuration. The second engagement portion 230 of the main body mounting portion 210 may be deformed or moved by elastic force. That is, the engagement state with the first engagement portion 180 of the main body portion 100 may be varied by deformation or movement of the second engagement portion 230 of the main body mounting portion 210 by elastic force.

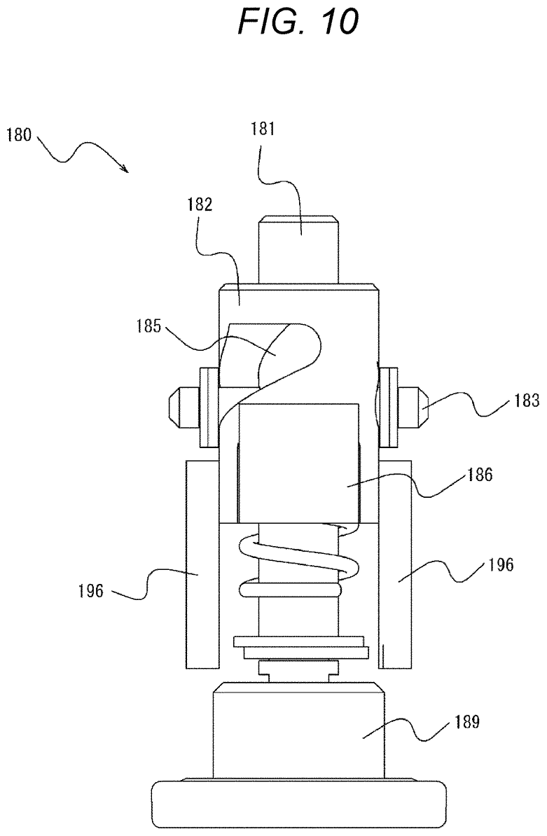

In the present embodiment, the endless portion 182 is provided with an elongated hole 185 inclined with respect to the longitudinal direction of the shaft 181. The shaft 181 is provided with a protrusion portion 183 that is inserted into the elongated hole 185 of the endless portion 182 and is movable along the extending direction of the elongated hole 185. Therefore, the endless portion 182 rotates relative to the shaft 181, in a manner that the endless portion 182 moves in the longitudinal direction of the shaft 181. Note that the movement of the claw portion 186 of the present embodiment in a plane orthogonal to the longitudinal direction of the shaft 181 is restricted by a restriction wall portion 196. FIG. 10 is an enlarged view of the first engagement portion 180 of the main body portion 100 as viewed from the distal end side of the claw portion 186. In FIGS. 6 to 9, only the restriction wall portion 196 positioned on the back side of the claw portion 186 is illustrated, but as illustrated in FIG. 10, the restriction wall portion 196 is disposed on both sides with the claw portion 186 sandwiched between. As a result, in the present embodiment, by inserting the second engagement portion 230 into the insertion hole 119 (see FIG. 4), mounting of the drug filling unit 200 to the main body portion 100 can be realized without further operation by the user.

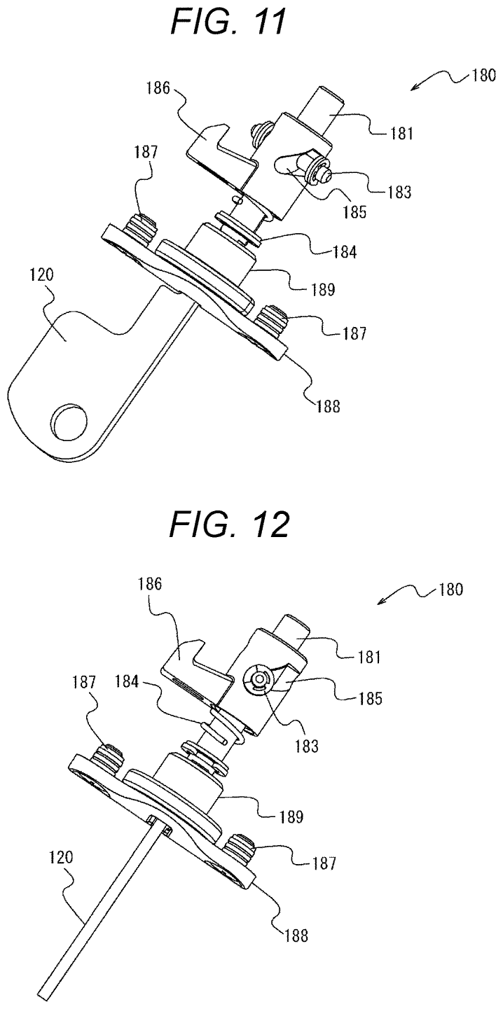

FIGS. 11 and 12 are enlarged perspective views of the first engagement portion 180 of the main body portion 100. In FIG. 11, the protrusion portion 183 is positioned on one end side (upper side and distal end side of the shaft 181 in FIGS. 11 and 12) of the elongated hole 185, while in FIG. 12, the protrusion portion 183 is positioned on the other end side (lower side and proximal end side of the shaft 181 in FIGS. 11 and 12.) of the elongated hole 185. By rotating the key 120 relative to the housing 195 of the main body portion 100, the shaft 181 also rotates in the same direction as the key 120. The endless portion 182 does not rotate together with the shaft 181 due to the above-described restriction wall portion 196. Therefore, the protrusion portion 183 attached to the shaft 181 slides on the inner surface of the elongated hole 185 inclined with respect to the shaft 181, pressing the endless portion 182 in the longitudinal direction of the shaft 181 and moving the endless portion 182 in the longitudinal direction of the shaft 181. As the endless portion 182 moves in the longitudinal direction of the shaft 181, the claw portion 186 attached to the endless portion 182 also moves in the longitudinal direction of the shaft 181. By using this mechanism, the first engagement portion 180 and the second engagement portion 230 are engaged and released. That is, when the endless portion 182 moves downward in the longitudinal direction of the shaft 181, the first engagement portion 180 and the second engagement portion 230 can be released.

As described in the preceding paragraphs, when the first engagement portion 180 and the second engagement portion 230 are engaged, the first engagement portion 180 moves downward by rotating the key 120 in a predetermined direction (counterclockwise with respect to the housing 195 of the main body portion 100 in the present embodiment). In addition, the drive unit cover 140 is biased in a direction toward the drug filling unit 200 by the elastic portion 142, and pushes back the drug filling unit 200 in a direction opposite to the moving direction at the time of mounting. As a result, in a state where the first engagement portion 180 moves downward and the engagement with the second engagement portion 230 can be released, the first engagement portion 180 is separated from the second engagement portion 230 against the restoring force of the elastic portion 184, and the engagement between the first engagement portion 180 and the second engagement portion 230 is released. After the engagement between the first engagement portion 180 and the second engagement portion 230 is released, the drug filling unit 200 pivots about the pivotal attachment portion 122 to be separated from the main body portion 100.

Referring again to FIG. 8, even after the first engagement portion 180 and the second engagement portion 230 is released, the ball plunger 190a as the third engagement portion 190 still remains in the opening of the second engagement portion 230. Therefore, even if the engagement between the first engagement portion 180 and the second engagement portion 230 is released and the first engagement portion 180 and the second engagement portion 230 are to be separated from each other, the second engagement portion 230 is engaged by the ball plunger 190a as the third engagement portion 190 (see FIG. 7). As a result, the drug filling unit 200 is not completely separated from the main body portion 100. In this manner, the half-latch mechanism between the main body portion 100 and the drug filling unit 200 is realized. Note that, because the ball plunger 190a as the third engagement portion 190 is movable or deformable in the longitudinal direction of the shaft 181, when the third engagement portion 190 moves or gets deformed in the longitudinal direction of the shaft 181, the engagement state between the second engagement portion 230 and the third engagement portion 190 is released. As a result, the drug filling unit 200 including the main body mounting portion 210 can be separated from the main body portion 100. For example, the half-latch mechanism can easily release the third engagement portion 190 by movement or deformation by applying force in a direction in which the main body portion 100 and the drug filling unit 200 are gripped and separated. With such a mechanism, in the infusion pump 1 having the main body portion 100 and the drug filling unit 200, a series of operations from the release of the engagement between the first engagement portion 180 and the second engagement portion 230 to the holding of the main body portion 100 and the drug filling unit 200 by the half-latch mechanism can automatically proceed only by the rotation operation of the key 120. In the present embodiment, the pivotal attachment portion 122 of the main body portion 100 is pivotally attached to the grip portion 250 of the drug filling unit 200, and the main body portion 100 and the drug solution filling unit 200 are stably held even in the holding state by the half-latch mechanism, and unexpected separation or falling can be further prevented. The infusion pump 1 can easily separate the main body portion 100 and the drug filling unit 200 by rotating the grip portion 250 with respect to the pivotal attachment portion 122 while gripping and applying a force to the main body portion 100 and the drug filling unit 200 after rotating the key 120.

As described above, when the endless portion 182 rotates relative to the shaft 181 and moves in the longitudinal direction of the shaft 181, the form of the first engagement portion 180 can be changed from the engagement state to the state in which the engagement can be released. The main body portion 100 includes the third engagement portion 190 that is fitted into an opening of the second engagement portion 230 to engage the second engagement portion 230 when the first engagement portion 180 and the second engagement portion 230 are brought into a state where the engagement can be released from the engagement state. The third engagement portion 190 allows the main body mounting portion 210 to be separated from the main body portion 100 by moving or being deformed in the longitudinal direction of the shaft 181.

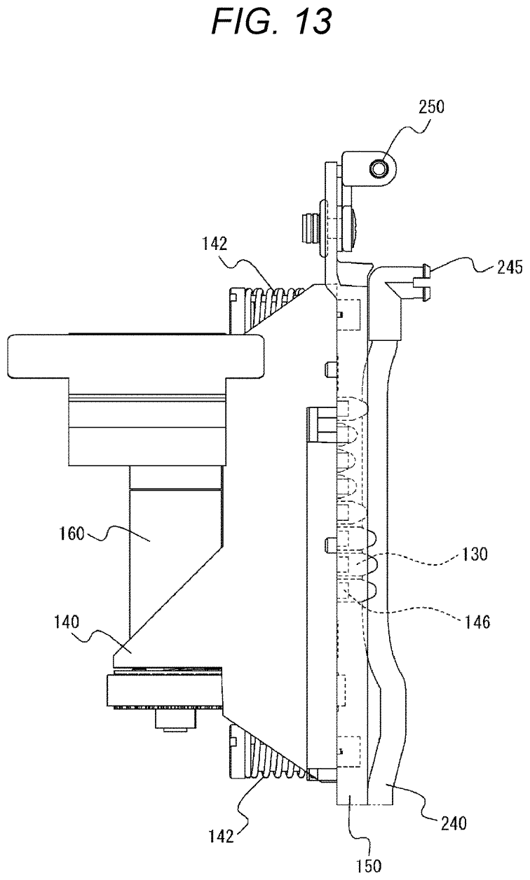

FIG. 13 illustrates a liquid feeding mechanism of the main body portion 100. The main body portion 100 further includes a chassis 150 that covers drive unit cover 140 and is fixed to the housing 195. The elastic portion 142 biases the drive unit cover 140 to come into contact with the chassis 150. Therefore, the drive unit 160 fixed to the drive unit cover 140 is also biased toward the chassis 150 by the elastic portion 142. An opening that enables contact between the finger portion 130 and the infusion tube 240 is provided in the chassis 150. Note that, in the present embodiment, the housing 195 and the chassis 150 are configured separately, but may be configured integrally.

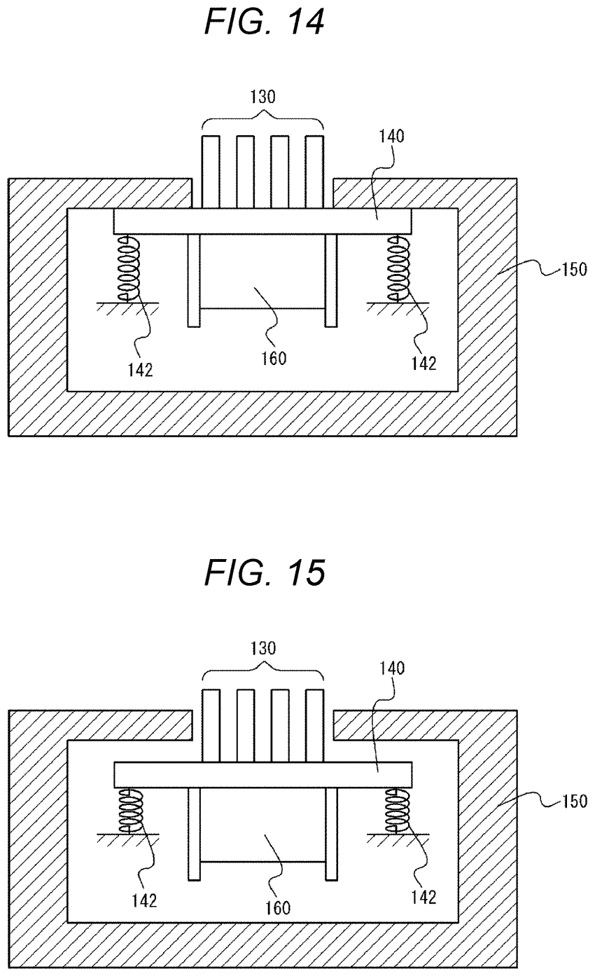

FIGS. 14 and 15 schematically illustrate a buffer mechanism of the infusion tube of the main body portion 100. The finger portions 130 are driven by the drive unit 160 that is mechanically connected, and are controlled by the control unit. When the infusion tube 240 is sandwiched between the main body portion 100 and the main body mounting portion 210, the finger portion 130 is pressed by the infusion tube 240. As a result, as shown in FIG. 15, the finger portions 130 are pushed down. Because the other finger portions 130 are connected to the drive unit cover 140, the elastic portions 142 bias the finger portions 130 to be pushed up. Therefore, the elastic portions 142 maintains a state in which the finger portions 130 abut on the infusion tube 240. As a result, it is possible to maintain a state in which the infusion tube 240 can be constantly pressed by the finger portions 130.

### Second Embodiment

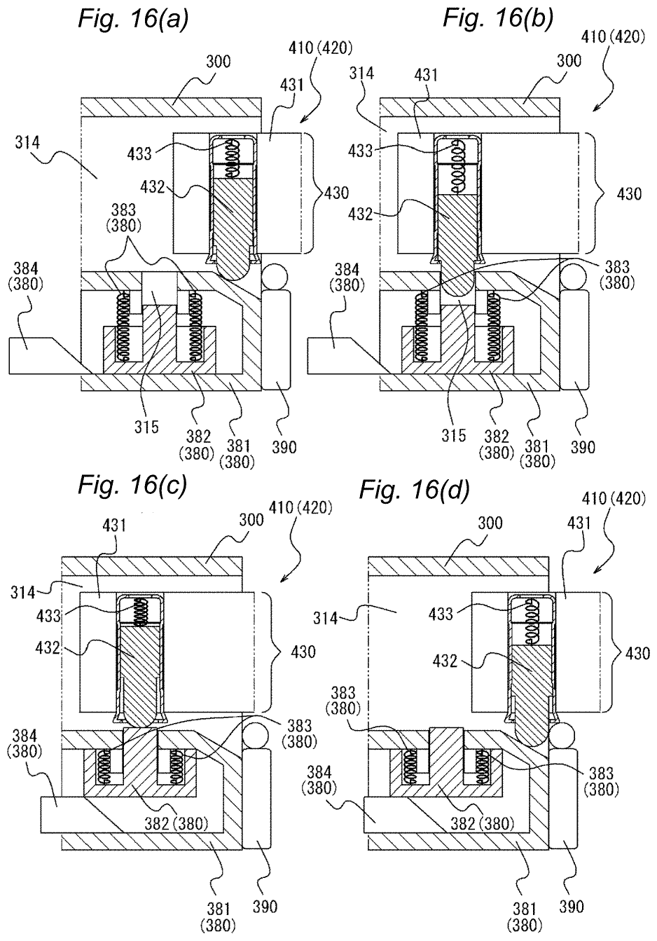

FIGS. 16(a)-16(d) illustrate an enlarged view of an engagement mechanism according to a second embodiment of the present application. An insertion hole 314 is formed in a main body portion 300, and the inlet of the insertion hole 314 is widened. The main body portion 300 includes a first engagement portion 380 and a third engagement portion 390 attached to the first engagement portion 380 or another portion of the main body portion 300 in the vicinity of the insertion hole 314. A main body mounting portion 410 of a drug filling unit 420 includes a second engagement portion 430. When the drug filling unit is mounted to the main body portion 300, the second engagement portion 430 is inserted into the insertion hole 314, and the first engagement portion 380 and the second engagement portion 430 are engaged with each other. In the present embodiment, the third engagement portion 390 is a ball plunger, but may be another engagement means.

The insertion hole 314 is formed above the first engagement portion 380. The first engagement portion 380 includes a housing 381, a movement engagement portion 382 provided in the housing 381, an elastic portion 383 that biases the movement engagement portion 382 toward the inner surface of the housing 381, and a pushing portion 384 that is provided adjacent to the movement engagement portion 382, is inserted between the movement engagement portion 382 and the inner surface of the housing 381, and can press the movement engagement portion 382 against the biasing force of the elastic portion 383. The second engagement portion 430 includes an insertion portion 431, a movement portion 432 provided in a concave portion extending in a direction (up-down direction in FIGS. 16(a)-16(d)) perpendicular to a moving direction (in the present embodiment, the direction is the same as the direction in which the insertion hole 314 extends in the insertion portion 431, and is the left direction in FIGS. 16(a)-16(d).) of the main body mounting portion 410 in the mounting operation, and an elastic portion 433 connecting the movement portion 432 and the insertion portion 431 and capable of biasing the movement portion 432 in a direction in which the movement portion 432 comes out of the concave portion.

In FIG. 16(a), the second engagement portion 430 is inserted into the insertion hole 314. More specifically, when the insertion portion 431 of the second engagement portion 430 approaches the inlet of the insertion hole 314 and comes into contact with the third engagement portion 390, the movement portion 432 pushes down the third engagement portion 390 by the elastic portion 433 and climbs over the third engagement portion 390. When the insertion portion 431 of the second engagement portion 430 climbs over the third engagement portion 390 and further enters the insertion hole 314, the third engagement portion 390 returns to the original position. After climbing over the third engagement portion 390, while the insertion portion 431 of the second engagement portion 430 further enters the insertion hole 314, the movement portion 432 is biased downward by the elastic portion 433 and moves while sliding with the housing 381.

FIG. 16(b) illustrates a state in which the insertion portion 431 of the second engagement portion 430 is further moved into the insertion hole 314 from the state illustrated in FIG. 16(a). As illustrated in FIG. 16(b), when the movement portion 432 reaches immediately above an opening portion 315 provided in the housing 381, the movement portion 432 is biased downward by the elastic portion 433, and thus the movement portion 432 enters the opening portion 315. As a result, the first engagement portion 380 and the second engagement portion 430 are engaged with each other. As described above, the first engagement portion 380 includes the housing 381 provided with the opening portion 315 extending in the direction perpendicular to the moving direction of the main body mounting portion in the mounting operation. The second engagement portion 430 includes an insertion portion 431 provided with a concave portion in a direction (up-down direction in FIGS. 16(a)-16(d)) perpendicular to the moving direction (left direction in FIGS. 16(a)-16(d)) of the main body mounting portion 410 in the mounting operation, and the movement portion 432 movable in the concave portion. The second engagement portion 430 is engaged with the first engagement portion 380 by the movement portion 432 varying the protrusion amount from the concave portion and entering the opening portion 315 of the housing 381 in the mounting operation.

In FIG. 16(c), when the pushing portion 384 approaches the movement engagement portion 382, the pushing portion 384 has a structure tapered toward the movement engagement portion 382, and thus moves below the movement engagement portion 382. That is, the pushing portion 384 is inserted between the movement engagement portion 382 and the inner surface of the housing 381. When the pushing portion 384 further approaches the movement engagement portion 382, because the pushing portion 384 has an inclined structure, the movement engagement portion 382 moves upward along the inclined surface of the pushing portion 384. As a result, the movement engagement portion 382 moves upward and pushes the movement portion 432 out of the opening portion 315. When the movement portion 432 completely comes out of the opening portion 315, the second engagement portion 430 becomes movable to come out of the insertion hole 314. As described above, the first engagement portion 380 is movable in the moving direction of the movement portion 432, and includes the movement engagement portion 382 engageable with the movement portion 432, and the pushing portion 384 capable of pushing out the movement engagement portion 382 toward the movement portion 432. The pushing portion 384 pushes out the movement engagement portion 382 toward the movement portion 432, and the movement portion 432 moves out of the opening portion 315 of the housing 381. Thus, the first engagement portion 380 and the second engagement portion 430 are released.

In FIG. 16(d), when the second engagement portion 430 moves toward the outside of the insertion hole 314 (toward the right side in FIGS. 16(a)-16(d)), the movement portion 432 is biased downward by the elastic portion 433, and thus slides on the housing 381 and moves. Because the third engagement portion 390 is provided to narrow the insertion hole 314, the movement portion 432 comes into contact with the third engagement portion 390. As a result, the second engagement portion 430 is engaged near the inlet of the insertion hole 314 by the third engagement portion 390. In this manner, a half-latch mechanism between the main body portion 300 and the main body mounting portion 410 of the drug filling unit 420 is realized. Note that, because the third engagement portion 390 is movable or deformable in the moving direction of the movement portion 432, when the third engagement portion 390 moves or gets deformed in the moving direction of the movement portion 432, the engagement state between the second engagement portion 430 and the third engagement portion 390 is released. As a result, the drug filling unit 420 including the main body mounting portion 410 can be separated from the main body portion 300. As described above, the main body portion 300 includes the third engagement portion 390 that is engaged with the second engagement portion 430 when the first engagement portion 380 and the second engagement portion 430 are brought into an engagement release state from an engagement state The third engagement portion 390 allows the drug filling unit 420 including the main body mounting portion 410 to be separated from the main body portion 300 by moving or being deformed in a moving direction (up-down direction in FIGS. 16(a)-16(d)) of the movement portion 432 with respect to the insertion portion 431.

In the present application, “engagement” means getting engaged with each other and stopped. In the embodiment described in the present application, the first engagement portion and the second engagement portion being engaged means that the first engagement portion and the second engagement portion are stopped by engagement between the first engagement portion and the second engagement portion, and as a result, the drug filling unit is mounted to the main body portion. In the embodiment described in the present application, the second engagement portion and the third engagement portion being engaged means that the second engagement portion is stopped by engagement between the second engagement portion and the third engagement portion, and as a result, the drug filling unit remains at a predetermined position. In addition, in the present application, the “half-latch” means a state in which the latch is not completely retained, but movement or displacement is limited.

### REFERENCE SIGNS LIST

1 Infusion pump
100 Main body portion
100a Main body facing surface
110 Operation unit
112 Display unit
113 Tube connector
114 Sensor hole
116 Finger portion hole
118 Key hole
119 Insertion hole
120 Key
122 Pivotal attachment portion
130 Finger portion
140 Drive unit cover
142 Elastic portion
144 Stick
146 Finger guide
150 Chassis
160 Drive unit
180 First engagement portion
181 Shaft
182 Cylindrical cam
183 Pin
184 Elastic portion
185 Elongated hole
186 Claw portion
187 Screw
188 Housing attachment portion
189 Shaft holding portion
190 Third engagement portion
190a Ball plunger
195 Housing
196 Restriction wall portion
200 Drug filling unit
210 Main body mounting portion
210a Tube receiving surface
220 Storage unit
230 Second engagement portion
240 Infusion tube
245 Tube connector
250 Grip portion
260 Tube guide portion
300 Main body portion
380 First engagement portion
381 Housing
382 Movement engagement portion
383 Elastic portion
384 Pushing portion
390 Ball plunger
430 Second engagement portion
431 Insertion portion
432 Movement portion
433 Elastic portion

1. An infusion pump for feeding a drug, the infusion pump comprising:
a main body portion comprising a first engagement portion; and
a main body mounting portion comprising a second engagement portion configured to engage with the first engagement portion; wherein:
the main body portion and the main body mounting portion are configured to feed a drug in an infusion tube by sandwiching the infusion tube therebetween;
the main body mounting portion is mounted on the main body portion by engaging the first engagement portion and the second engagement portion; and
the first engagement portion and the second engagement portion are engaged with each other by deformation or movement of the first engagement portion or the second engagement portion by elastic force in conjunction with a mounting operation in which the main body portion and the main body mounting portion approach each other with the infusion tube sandwiched therebetween.

2. The infusion pump according to claim 1, wherein the first engagement portion or the second engagement portion is configured to be deformed or moved by an elastic force in a direction intersecting a moving direction of the main body portion and the main body mounting portion in the mounting operation.

3. The infusion pump according to claim 1, wherein:
one of the first engagement portion or the second engagement portion comprises a claw portion configured to engage with the other of the first engagement portion or the second engagement portion, an endless portion to which the claw portion is attached, a shaft penetrating the endless portion, and an elastic portion configured to bias the endless portion in a longitudinal direction of the shaft; and
the one engagement portion is configured to engage with the other engagement portion by the claw portion and the endless portion moving in the longitudinal direction of the shaft by an elastic force of the elastic portion.

4. The infusion pump according to claim 3, wherein:
an elongated hole extending in a direction inclined with respect to the longitudinal direction is formed in the endless portion;
the shaft includes a protrusion portion inserted into the elongated hole; and
the endless portion is configured to move in the longitudinal direction by rotating relative to the shaft.

5. The infusion pump according to claim 4, wherein:
the first engagement portion and the second engagement portion are configured to be changed from an engagement state to an engagement release state by the endless portion rotating relative to the shaft and moving in the longitudinal direction;
the main body portion comprises a third engagement portion that engages the second engagement portion when the first engagement portion and the second engagement portion are changed from the engagement state to the engagement release state; and
the third engagement portion allows the main body mounting portion to be separated from the main body portion by moving or being deformed in the longitudinal direction.

6. The infusion pump according to claim 1, wherein:
the first engagement portion includes a housing provided with an opening portion extending in a direction perpendicular to a moving direction of the main body mounting portion in the mounting operation;
the second engagement portion comprises:
an insertion portion comprising a concave portion that is concave in a direction perpendicular to the moving direction, and
a movement portion having a protrusion amount from the concave portion that is variable by moving in the concave portion; and
the second engagement portion is configured to engage with the first engagement portion by the movement portion varying a protrusion amount from the concave portion in the mounting operation and entering the opening portion of the housing.

7. The infusion pump according to claim 6, wherein:
the first engagement portion further comprises:
a movement engagement portion that is movable in a moving direction of the movement portion and is engageable with the movement portion, and
a pushing portion configured to push out the movement engagement portion toward the movement portion; and
the first engagement portion and the second engagement portion are released by the pushing portion pushing out the movement engagement portion toward the movement portion and the movement portion moving out of the opening portion of the housing.

8. The infusion pump according to claim 7, wherein:
the main body portion comprises a third engagement portion that engages the second engagement portion when the first engagement portion and the second engagement portion are changed from an engagement state to an engagement release state; and
the third engagement portion allows the main body mounting portion to be separated from the main body portion by moving or being deformed in the moving direction of the movement portion.

9. The infusion pump according to claim 1, wherein:
the endless portion is a cylindrical portion.

10. An infusion pump for feeding a drug, the infusion pump comprising:
a main body portion comprising a first engagement portion, wherein the first engagement portion comprises:
a claw portion,
a cylindrical portion to which the claw portion is attached,
a shaft penetrating the cylindrical portion, and
an elastic portion configured to bias the cylindrical portion in a longitudinal direction of the shaft; and
a main body mounting portion comprising a second engagement portion configured to engage with the first engagement portion, wherein the second engagement portion comprises:
a protrusion having an opening; wherein:
the main body portion and the main body mounting portion are configured to feed a drug in an infusion tube by sandwiching the infusion tube therebetween;
the main body mounting portion is mounted on the main body portion by engaging the first engagement portion and the second engagement portion; and
the first engagement portion and the second engagement portion are engaged with each other by deformation or movement of the first engagement portion or the second engagement portion by elastic force in conjunction with a mounting operation in which the main body portion and the main body mounting portion approach each other with the infusion tube sandwiched therebetween.

11. A method of operating an infusion pump for feeding a drug, the method comprising:
providing the infusion pump, which comprises:
a main body portion comprising a first engagement portion; and
a main body mounting portion comprising a second engagement portion configured to engage with the first engagement portion;
performing a mounting operation in which the main body portion and the main body mounting portion approach each other with the infusion tube sandwiched therebetween, thereby causing the first engagement portion and the second engagement portion to engage with each other by deformation or movement of the first engagement portion or the second engagement portion by elastic force, and thereby mounting the main body mounting portion on the main body portion; and
feeding a drug in the infusion tube sandwiched between the main body portion and the main body mounting portion.
